(19) United States
(12) Patent Application Publication
(10) Pub. No.: US 2012/0114564 A1
(43) Pub. Date: May 10, 2012

(54) MRI T1 CONTRASTING AGENT COMPRISING MANGANESE OXIDE NANOPARTICLE

(75) Inventors: Taeghwan Hyeon, Seoul (KR); Kwangjin An, Seoul (KR); Hyon Bin Na, Seoul (KR); Junghee Lee, Seoul (KR)

(73) Assignee: SEOUL NATIONAL UNIVERSITY INDUSTRY FOUNDATION, Seoul (KR)

(21) Appl. No.: 12/525,276

(22) PCT Filed: Jan. 30, 2008
(86) PCT No.: PCT/KR08/00574

§ 371 Date: Nov. 9, 2009

(30) Foreign Application Priority Data
Jan. 30, 2007 (KR) 10-2007-0009707
Jul. 31, 2007 (KR) 10-2007-0077029

(51) Int. Cl. A61K 49/12; A61K 49/16; C01G 45/02; A61K 49/10; A61K 49/14; B82Y 5/00; B82Y 40/00

(52) U.S. Cl. 424/9.322; 424/9.32; 423/605; 428/402; 977/811; 977/890; 977/930

(57) ABSTRACT

The present invention relates to the use of and method for using MnO nanoparticles as MRI T1 contrasting agents which reduces T1 of tissue. More specifically, the present invention is directed to MRI T1 contrasting agent comprising MnO nanoparticle coated with a biocompatible material bound to a biologically active material such as a targeting agent, for example tumor marker etc., and methods for diagnosis and treatment of tumor etc. using said MRI T1 contrasting agent, thereby obtaining more detailed images than the conventional MRI T1-weighted images. The MRI T1 contrasting agent of the present invention allows a high resolution anatomic imaging by emphasizing T1 contrast images between tissues based on the difference of accumulation of the contrasting agent in tissues. Also, the MRI T1 contrasting agent of the present invention enables to visualize cellular distribution due to its high intracellular uptake. The MRI T1 contrasting agent of the present invention can be used for target-specific diagnosis and treatment of various diseases such as tumor etc. when targeting agents binding to disease-specific biomarkers are conjugated to the surface of nanoparticles.

### TECHNICAL FIELD

The present invention relates to the use of and method for using MnO nanoparticles as MRI T1 contrasting agents which reduces T1 of tissue. More specifically, the present invention is directed to MRI T1 contrasting agent comprising MnO nanoparticle coated with a biocompatible material bound to a biologically active material such as a targeting agent, for example tumor marker etc., and methods for diagnosis and treatment of tumor etc. using said MRI T1 contrasting agent, thereby obtaining more detailed images than the conventional MRI T1-weighted images.

The MRI T1 contrasting agent of the present invention allows a high resolution anatomic imaging by emphasizing T1 contrast images between tissues based on the difference of accumulation of the contrasting agent in tissues. Also, the MRI T1 contrasting agent of the present invention enables to visualize cellular distribution due to its high intracellular uptake. The MRI T1 contrasting agent of the present invention can be used for target-specific diagnosis and treatment of various diseases such as tumor etc. when targeting agents binding to disease-specific biomarkers are conjugated to the surface of nanoparticles.

### BACKGROUND ART

Magnetic Resonance Imaging (MRI), one of the most potent diagnostic imaging techniques, utilizes the spin relaxation of the hydrogen atom in a magnetic field to obtain anatomical, biological, and biochemical information as images through real-time non-invasive imaging of organs of living humans and animals.

A contrasting agent of the present invention refers to a material which enhances image contrast by injecting said contrasting agent into a living organism in order to utilize MRI extensively and precisely in the applications of bioscience and medical science. The contrast between tissues in MRI images arises since the relaxation that the nuclear spin of water molecules in the tissues returns to its equilibrium state differs from each other. Contrasting agents have an influence on the relaxation thereby widening the difference of relaxitivity between the tissues and induces change in the MRI signal thereby creating a more distinct contrast between tissues.

The difference of applicability and preciseness of a contrasting agent arises due to characteristic and function thereof and the subject injected therewith. Enhanced contrast provided by a contrasting agent allows image signals of a specific living organ and surroundings of tissues to be clearly visualized by increasing or decreasing the image signals. A ‘positive’ contrasting agent refers to a contrasting agent that enhances the image signals of the desired body part for MRI imaging relative to its surroundings, and a ‘negative’ contrasting agent, vice versa.

A positive contrasting agent is a contrasting agent relating to T1 relaxation, or longitudinal relaxation. The longitudinal relaxation is a process by which z component of the nuclear spin magnetization, M_{z}, in a non-equilibrium state caused by absorbing RF energy exerted in the direction of x-axis aligns on y-axis on the x-y plane and then returns to equilibrium state by releasing the absorbed RF energy. The longitudinal relaxation is also called “T1 relaxation”. T1 relaxation time is time after which M_{z} recovers to 63% of its equilibrium value. As T1 relaxation time shortens, MRI signals increases and, thus, the image acquisition time decreases.

A negative agent is a contrasting agent relating to T2 relaxation, or transverse relaxation. T2 relaxation refers to a phenomenon that y component of the nuclear spin magnetization which widened uniformly on the x-y plane, M_{y}, decays exponentially while M_{z} in a non-equilibrium state caused by absorbing RF energy exerted in the direction of x-axis aligns on y-axis on the x-y plane and then returns to equilibrium state by releasing the absorbed RF energy to the surrounding spins. T2 relaxation time is time after which M_{y} drops to 37% of its original magnitude. A function of time which describes that M_{y} decreases dependent on time, and is measured through a receiver coil installed on the y-axis is called free induction decay (FID) signal. Tissue with short T2 time appears dark in the MRI image.

Paramagnetic complexes for positive contrasting agents and superparamagnetic nanoparticles for negative contrasting agents, which have been currently commercialized, are being used for MRI contrasting agents. The paramagnetic complexes, positive contrasting agents, that are usually gadolinium (Gd³⁺) or manganese (Mn²⁺) chelates, accelerate longitudinal (T1) relaxation of water proton and exert bright contrast in regions where the complexes localize.

However, Gadolinium ion is very toxic, and thus in order to prevent this, Gadolinium ion is used in the form of a chelate or a polymer-bound compound. Amongst, Gd-DTPA has been most widely used and its main clinical applications are focused on the detection of the breakage of blood brain barrier (BBB) and changes in vascularity, flow dynamics and perfusion. The contrasting agents trigger the immune system of a living organism or decompose in the liver since said contrasting agents are in the form of a compound. Thus, the contrasting agents causes said contrasting agents to reside in blood for a short period of time, about 20 minutes.

Manganese-enhanced MRI (MEMRI) using manganese ion (Mn²⁺) as a T1 contrast agent has been used for imaging anatomic structures and cellular functions in a wide variety of brain science research etc. (Lin Y J, Koretsky A P, Manganese ion enhances T1-weighted MRI during brain activation: an approach to direct imaging of brain function, Magn. Reson. Med. 1997; 38: 378-388) Despite the excellent properties of Mn²⁺ as a contrast agent for MEMRI, it has been applicable only for contrasting of animal brains with a large dose (>88˜175 mg/kg) delivered in the form of MnCl₂ due to the toxicity of Mn²⁺ ions when they accumulate excessively in tissues. Consequently, MEMRI has intrinsic limitations to be further developed for human brain application.

A contrasting agent using manganese ions, Mn-DPDP (teslascan), is currently known to the public, which is used for contrasting the human liver. When Mn-DPDP is administered into the body, Zn²⁺ replaces Mn²⁺ to become Zn-DPDP and is excreted through the kidney, and the Mn²⁺ acts as a contrasting agent as it circulates through the blood and is absorbed by the liver, kidney, pancreas, etc. Due to the toxicity of Mn²⁺, a slow infusion, approximately 2 to 3 ml/hr, is required. Ordinarily, approximately 5 μmol/kg (0.5 ml/kg) can be administered to humans, however this amount is completely insufficient for contrasting the brain or other organs (ref. Rofsky N M, Weinreb J C, Bernardino M E et al. Hepatocellular tumors: characterization with Mn-DPDP-enhanced MR imaging. Radiology 188:53, 1993).

T1 contrast which uses positive contrasting agents, do not produce distortions in images, and is suitable for researching the anatomic structures in tissues and the function of cells. Also, T1 contrast is the most widely used in MRI due to high resolution images and thus are being extensively researched and developed. However, the conventional positive contrasting agents have limitations in human application since the conventional positive contrasting agents composed of paramagnetic metal ions for derivatives thereof are toxic. Also, the conventional positive contrasting agents have a short residence time in blood. Furthermore, it is difficult to conjugate targeting agents with he conventional positive contrasting agents due to steric hindrance of the ligand of the complex.

In order to overcome the above-mentioned problems, US 2003/0215392 A1 discloses polymer nanostructures enriched with gadolinium ions so as to increase local concentration of said nanostructures and maintain the shape of said nanostructures. However, due to the large size of the polymer nanostructures and the state in which the gadolinium ion is bound to the polymer nanostructure, the gadolinium ion can be easily separated from the surface of the nanostructure. Also, the polymer nanostructures show a low degree of intracellular uptake.

Superparamagnetic nanoparticles are used for negative contrasting agents, of which superparamagnetic iron oxide (SPIO) is the representative example.

U.S. Pat. No. 4,951,675 discloses a MRI T2 contrasting agent using a biocompatible superparamagnetic particle and U.S. Pat. No. 6,274,121 discloses a superparamagnetic particles consist of superparamagnetic one-domain particles and aggregates of superparamagnetic one-domain particles to whose surfaces are bound inorganic and optionally organic substances optionally having further binding sites for coupling to tissue-specific binding substances, diagnostic or pharmacologically active substances.

SPIO nanoparticles are nanometer-sized and thus reside in a living organism for hours. Also, a variety of functional groups and targeting materials can be conjugated to the surface of the SPIO nanoparticle. Thus, the SPIO nanoparticles have been the prevailing target-specific contrasting agent.

However, the inherent magnetism of the SPIO nanoparticle shortens its T2 relaxation time, and thus produces the magnetic field which distorts MRI image. In addition, the dark region in T2 weighted MRI, which results from the shortened T2 relaxation time, is often confused with the intrinsically dark region originated from, for example, internal bleeding, calcification or metal deposits.

Moreover, the inherent magnetism of the SPIO nanoparticle causes a blooming effect on the magnetic field near the SPIO nanoparticle and thus produces signal loss or distortions in the background image, which makes it impossible to obtain the proximate anatomical images.

### DISCLOSURE

### Technical Problem

Therefore, the object of the present invention is to provide an MRI T1 contrasting agent comprising manganese oxide (MnO) nanoparticle, which produces brightened and undistorted T1 contrast effects due to Mn²⁺ ions on the surface of the MnO nanoparticles, and satisfies high intracellular uptake and accumulation resulted from nanoparticulate form, target-specific contrast ability, easy delivery, and safe clearance from patients with minimal side effects.

The nanoparticulate T1 contrasting agent of the present invention lengthens the period of time for its residence in a living organism compared with the conventional T1 contrasting agents based on gadolinium or manganese in the form of ions or complexes, and thus it is possible to secure a sufficient time for an MRI scan and diagnosis after injecting the contrast agent. Also, the T1 contrasting agent of the present invention resides in a cell due to the high intracellular uptake, which makes it possible to obtain continuous or intermittent diagnostic imaging for an extended period of time and cellular imaging at the level of a cell.

Another object of the present invention is to provide a method for preparing a MRI T1 contrasting agent, comprising:

i) thermolyzing a Mn—C₄₋₂₅ carboxylate complex to prepare a manganese nanoparticle with a diameter not exceeding preferably 50 nm, more preferably 40 nm, most preferably 35 nm, dispersed in an organic solvent selected from the group consisting of C₆₋₂₆ aromatic hydrocarbon, C₆₋₂₆ ether, C₆₋₂₅ aliphatic hydrocarbons, C₆₋₂₆ alcohol, C₆₋₂₆ thiol, and C₆₋₂₅ amine; and ii) coating said manganese oxide nanoparticle with a biocompatible material.

Yet another objet of the present invention is to provide an MRI T1 contrasting agent comprising manganese oxide (MnO) nanoparticle, a biocompatible material and a biologically active material, said manganese oxide nanoparticle being coated with said biocompatible material conjugated with said biologically active material.

Therefore, the present invention provides a composition for diagnosis or treatment, which contains targeting agents such as a tumor marker, etc. and a biologically acceptable carrier by introducing adhesive regions or reactive regions to the MnO nanoparticle.

Yet another objet of the present invention is to provide a method for MRI T1 contrasting for animal cells using a MRI T1 contrasting agent comprising Manganese Oxide (MnO) nanoparticles.

Yet another objet of the present invention is to provide a method for MRI T1 contrasting for animal blood vessels using a MRI contrasting agent comprising Manganese Oxide (MnO) nanoparticles.

### Technical Solution

The object of the present invention can be achieved by providing an MRI T1 contrasting agent comprising manganese oxide (MnO) nanoparticle.

The “MnO nanoparticles” of the present invention refers to nanoparticles which comprise MnO or a multi-component hybrid structure and have the diameter of preferably no more than 1,000 nm, more preferably no more than 100 nm.

The size of MnO nanoparticles suitable for the MRI contrasting agent of the present invention is preferably no more than 50 nm, more preferably no more than 35 nm, and most preferably no more than 30 nm. Also, the standard deviation of diameter variation of the MnO nanoparticles for the MRI contrasting agent of the present invention is preferably no more than 15%, more preferably no more than 10%, and most preferably no more than 5%.

The range of the sizes of the MnO nanoparticles of the present invention is not only a technical feature to produce continuous or intermittent MRI imaging, the MnO nanoparticles remaining in blood vessels, but also a technical element to keep an MnO nanoparticles-dispersed aqueous solution stable.

Therefore, the present invention is accomplished by the technical feature that the size of the MnO nanoparticles used for the MRI contrasting agent of the present invention can be controlled to be no more than a required size, most preferably no more than 35 nm.

The conventional T1 contrasting agent, specifically the T1 contrasting agent based on Mn²⁺ is toxic to a human due to the competition of Mn²⁺ with Ca²⁺. However, according to the MnO nanoparticles of the present invention, manganese forms solid particle and therefore the MnO nanoparticle of the present invention is almost non-toxic.

Also, in order to be used for a contrasting agent for cells and blood vessels, the MnO MRI contrasting agent of the present invention can be stabilized in dispersion in blood by coating the contrast agent with a biocompatible material and thus easily permeate in vivo membranes including a cell membrane.

The diameter of the MRI T1 contrasting agent of the present invention, in the state of being coated with a biocompatible material, is no more than 500 nm, preferably no more than 100 nm, most preferably no more than 50 nm. The size varies depending upon the coating material and, for example, the size can exceed 100 nm when coated with dextran. However, the degradation of the contrasting agent by the immune system or a liver can be minimized by reducing the size of the contrasting agent, preferably no more than 100 nm. Thereby, one of the technical features of the present invention is that the continuous or intermittent MRI imaging for a period of extended time can be made.

As described above, the MnO nanoparticles of the present invention can be used for T1 contrasting agent having as excellent T1 contrast effect as the conventional T1 contrasting agent based on Mn²⁺, resulting from manganese in the MnO nanoparticle. The chemical formula of the manganese oxide nanoparticle is MnO, and the manganese ions of the MnO nanoparticle have a T1 contrast effect in the way of accelerating the spins of water molecules surrounding said MnO nanoparticles.

The MnO nanoparticles of the present invention is antiferromagnetic and is not magnetized at ambient temperature. Therefore, the MnO nanoparticles of the present invention do not produce signal loss and distortion in images caused by the self-magnetization as SPIO.

Since the MnO nanoparticles of the present invention have a size no more than a certain value, the MnO nanoparticle shows high intracellular uptake and accumulation, and can be used for an MRI contrasting agent which may be conjugated with active materials such as targeting agents in a living organism.

The MRI contrasting agent comprising MnO nanoparticles of the present invention is stably dispersed in aqueous solution, easily coated with biocompatible materials, comprising a reactive region binding to in vivo active component such as targeting agents, and suitable for the diagnostic or treating agent for diseases.

Another object of the present invention can be achieved by providing a method for preparing a MRI T1 contrasting agent, comprising:

i) thermolyzing a Mn—C₄₋₂₅ carboxylate complex to prepare a manganese nanoparticle with a diameter preferably not exceeding 50 nm, more preferably not exceeding 40 nm, and most preferably not exceeding 35 nm, dispersed in an organic solvent selected from the group consisting of C₆₋₂₆ aromatic hydrocarbon, C₆₋₂₆ ether, C₆₋₂₅ aliphatic hydrocarbons, C₆₋₂₆ alcohol, C₆₋₂₆ thiol, and C₆₋₂₅ amine; and

ii) coating said manganese oxide nanoparticle with a biocompatible material.

It should be appreciated by a person skilled in the art that all MnO nanoparticles prepared by the conventional methods can be used for the contrasting agent of the present invention, although the conventional methods were not described herein.

The biocompatible material of the step ii) is selected from polyvinyl alcohol, polylactide, polyglycolide, poly(lactide-co-glycolide), polyanhydride, polyester, polyetherester, polycaprolactone, polyesteramide, polyacrylate, polyurethane, polyvinyl fluoride, poly(vinyl imidazole), chlorosulphonate polyolefin, polyethylene oxide, poly(ethylene glycol), dextran, the mixtures thereof or the copolymers thereof, which are non-toxic in vivo.

It should be understood by a person skilled in the art that all the conventional materials which are blood- or bio-compatible can be used for the contrasting agent of the present invention, although the conventional materials were not described herein.

Yet another object of the present invention can be achieved by providing an MRI T1 contrasting agent comprising manganese oxide (MnO) nanoparticle, a biocompatible material and a biologically active material, said manganese oxide nanoparticle being coated with said biocompatible material conjugated with said biologically active material.

The biologically active material is selected from an antibody comprising an antibody which selectively conjugates to a target material in a living organism, a monoclonal antibody prepared by the above antibody, variable region or constant region of an antibody, a chimeric antibody of which sequence is changed partly or wholly, a humanized chimeric antibody, etc.; a targeting agent comprising nucleic acids such as RNA or DNA which has a sequence complimentary to a specific RNA or DNA, non-biological compounds which can bind to a specific functional group via, for example, a hydrogen bonding, etc.; a medicinally active material; an apoptosis-inducing gene or a toxic protein; fluorescent material; a material which is sensitive to light, electromagnetic wave, radiation or heat; isotope.

The biologically active materials which can be conjugated with the MnO nanoparticle MRI contrasting agent of the present invention include other conventional biologically active materials and there is no limitation.

More particularly, the biologically active materials which can be conjugated with the MnO nanoparticles of the present invention, comprise all the biologically active materials currently known to the public, and there is no limitation on biologically active material. However, the above-mentioned biologically active materials, used for a cell contrasting agent, are limited to materials which have a cell membrane permeability equal to that of the MnO nanoparticles of the present invention.

As described above, the materials which can be conjugated with the MnO nanoparticles of the present invention and the method for conjugation therebetween are disclosed by, for example, U.S. patent application Ser. Nos. 11/410,607, 11/335,995, 11/171,761, 10/640,126, 11/348,609 and 10/559,957, which are incorporated herein by reference.

The MnO nanoparticles of the present invention can be conjugated with active materials such as a medicinally active material, a material which is sensitive to light, electromagnetic wave, radiation or heat. Specifically, the MnO nanoparticles can be conjugated with materials which can diagnose and/or treat tumors, specific proteins, etc. The biologically active material conjugated MnO nanoparticles of the present invention can be used for the diagnosis and/or treatment of various tumor-related diseases such as gastric cancer, lung cancer, breast cancer, hepatoma, laryngeal cancer, cervical cancer, ovarian cancer, bronchial cancer, nasopharyngeal cancer, pancreatic cancer, bladder cancer, colon cancer, etc., and specific protein-related diseases such as Alzheimer's disease, Parkinson's disease, bovine spongiform encephalopathy, etc.

These tumors or specific proteins secrete and/or express specific materials which are not secreted or expressed by normal cells and proteins. The specific materials are conjugated with the biologically active materials of the MnO nanoparticles of the present invention and then used for the diagnosis and/or treatment of the above-mentioned diseases.

The biologically active materials which can be conjugated with the MnO nanoparticles of the present are listed in Table 1 and, however, the biologically active materials are not limited thereto.

TABLE 1
Targeting agents

| types | desease | targeting agents |
|---|---|---|
| antibodies | non-Hodgkin lymphoma | Rituxan |
| | breast cancer | Herceptin |
| | immunorejection | Orthoclone |
| | arteriosclerosis | Reopro |
| | immunorejection | Zenapax |
| | respiratory desease | Synagis |
| | rheumatism, inflammatory desease | Remicade |
| | immunorejection | Mylotarg |
| | leukemia | Campath |
| | lung cancer, colon cancer | Erbitux |
| | lung cancer, colon cancer, breast cancer | Avastin |
| | malignant lymphoma | Zevalin |
| | non-Hodgkin lymphoma | Bexxar |
| receptor | ovarian cancer | folic acid |
| ligands | tumors | VEGFR |
| | | EGFR |
| peptide | Alzheimer's desease | Abeta |

That is, the biologically active material is selected from Rituxan, Herceptin, Orthoclone, Reopro, Zenapax, Synagis, Remicade, Mylotarg, Campath, Erbitux, Avastin, Zevalin, Bexxar, or the mixtures thereof, etc.; folic acid, Vascular Endothelial Growth Factor Receptor (VEGFR), Epidermal Growth Factor Receptor (EGFR), or the ligands thereof; amyloid beta peptide (Abeta), peptide containing RGD (Arg-Gly-Asp) amino acid sequence, nuclear localization signal (NLS) peptide, TAT protein or the mixtures thereof. The MnO nanoparticles of the present invention can be conjugated with either any material which allows targeting and treating simultaneously, or an therapeutic agent such as an anticancer drug.

Currently, a variety of the conventional therapeutic agents related tumors and specific proteins can be used for a method for treatment of the aforementioned diseases, which are selected from cisplatin, carboplatin, procarbazine, cyclophosphamide, dactinomycin, daunorubicin, doxorubicin, bleomycin, taxol, plicamycin, mitomycin, etoposide, tamoxifen, transplatinum, vinblastin, methotrexate, etc., but not limited thereto.

Yet another object of the present invention can be achieved by providing a method for MRI T1 contrasting for animal cells using a MRI T1 contrasting agent comprising Manganese Oxide (MnO) nanoparticles.

That is, the present invention provides a method for diagnosis or treatment of the aforementioned diseases, comprising: i) administrating the MRI T1 contrasting agent comprising the MnO nanoparticles of the present invention to a living organism or a sample to obtain T1 weighted MR images therefrom; ii) administrating the MRI T1 contrasting agent comprising the MnO nanoparticles conjugated with targeting agents and/or therapeutic agents, to a living organism or a sample to obtain T1 weighted MR images therefrom; and iii) sensing, via a diagnostic equipment, the signals produced by the MRI T1 contrasting agent comprising MnO nanoparticles to diagnose tissues.

The route of administration of the MRI T1 contrasting agent of the present invention may be preferably parenteral, for example, intravenous, intraperitoneal, intramuscular, subcutaneous or topical.

After the administration of the MRI T1 contrasting agent comprising MnO nanoparticles, the diagnostic method uses a diagnostic equipment including an MRI system. Diagnosis can be performed with a diagnostic equipment including the conventional MRI system using a magnetic field intensity of 1.5T, 3T, 4.7T, 9T, etc. The method for MR imaging by using MnO nanoparticles may be performed by a diagnostic method using T1 weighted images and also be carried out by diagnostic methods using both T1 weighted images and T2 weighted images.

Anatomical information, at cellular levels, between normal and abnormal tissues can be obtained from images of living organs or samples including brain, bone marrow, joint, muscles, liver, kidney, stomach, etc., produced by a diagnostic equipment using MRI T1 contrasting agent comprising the MnO nanoparticles.

The existence of a target can be seen from images produced by a diagnostic MRI equipment using the targeting and/or biologically active materials carried MnO nanoparticles. The distribution of the targets makes it possible to diagnose the progression of tumors, specific proteins, etc. In addition, the localization of therapeutic agents carried by the MnO nanoparticles makes it possible to treat said tumors, specific proteins, etc.

Yet another object of the present invention can be achieved by providing a method for MRI T1 contrasting for animal blood vessels using a MRI contrasting agent comprising Manganese Oxide (MnO) nanoparticles. The MnO nanoparticles used for MRI T1 contrasting for animal blood vessels, have weaker limitations on the size than the cell contrasting agent in that the blood vessel contrasting agent is not strongly required a cell membrane permeability, comparing with the cell contrasting agent. However, much great size of the blood vessel contrasting agent causes the activation of the immune system or the degradation in liver, which still has a disadvantage of the decrease in residence time of the contrasting agent in blood vessels.

### Advantageous Effects

Firstly, the MnO nanoparticles according to the present invention make it possible to produce bright T1 weighted imaging of various organs such as brain, liver, kidney, spinal cord, etc.; to visualize anatomic structures of brain due to high intracellular uptake, particularly due to the passage through blood brain barrier (BBB); and to image human cells and blood vessels by removing the toxicity of Mn²⁺.

Secondly, the conjugation of the MnO nanoparticle with targeting agents allows the target imaging of cells such as cancer, tumors, etc.; monitoring of expression and migration of cells such as stem cells, in cytotherapy since it is easy to modify the surface of the MnO nanoparticles of the present invention.

### DESCRIPTION OF DRAWINGS

FIG. 1 shows TEM images of water-dispersible MnO nanoparticles of the present invention with various particle sizes.

FIG. 2 shows a magnetization curve of the MnO nanoparticles of the present invention at ambient temperature.

FIG. 3 shows T1 weighted MRI of the MnO nanoparticles of the present invention with various particle sizes at 3.0 T clinical MRI system.

FIG. 4 shows T1 weighted manganese oxide nanoparticle enhanced MRI (MONEMRI) of brain of a mouse before and after the injection of the MnO nanoparticles of the present invention to the mouse through a vein.

FIG. 5 shows T1 weighted MONEMRI of kidney (A), liver (B) and spinal cord (C) before and after the injection of the MnO nanoparticles of the present invention to the mouse through a vein.

FIG. 6 shows MONEMRI of a gliblastoma tumour bearing mouse brain.

FIG. 7 shows T1 weighted MRI images of a mouse brain which bears a breast cancer brain metastatic tumor, with a functionalized MnO nanoparticles by conjugation with Her-2/neu (Herceptin), and with a non-functionalized MnO nanoparticles.

FIG. 8 shows hydrodynamic diameters of the DNA conjugated MnO nanoparticles of the present invention, measured by dynamic light scattering.

FIG. 9 shows results of electrophoresis of MnO nanoparticles and DNA conjugated MnO nanoparticles.

FIG. 10 shows results of electrophoresis of DNA, DNA conjugated with MnO nanoparticle, and released DNA after DTT treatment.

### BEST MODE

Hereinafter, the present invention will be described in greater detail with reference to the following examples. The examples are given only for illustration of the present invention and not to be limiting the present invention.

### Example 1

### Preparation of MnO Nanoparticles Coated with Biocompatible Materials

A variety of methods can produce MnO nanoparticles coated with biocompatible materials. An exemplary method for preparing MnO nanoparticles coated with biocompatible materials is as follows, but not limited to the MnO nanoparticles prepared thereby.

Therefore, the particle size of the blood vessel contrasting agent of the present invention is preferably no more than 500 nm, and more preferably no more than 100 nm. The MnO MRI contrasting agent of the present invention, used for contrasting animal blood vessels, may be preferably dispersed into a blood-compatible material such as dextran.

At first, Mn-oleate complexes were synthesized. 7.92 g of manganese chloride tetrahydrate and 24.36 g of sodium oleate were added to a mixture composed of ethanol, distilled water, and n-hexane. The resulting mixture solution was heated to 70° C. and maintained overnight at this temperature. The solution was then transferred to a separatory funnel and the upper organic layer containing the Mn-oleate complex was washed several times using distilled water. The evaporation of the hexane solvent produced a pink coloured Mn-oleate powder.

Then, MnO nanoparticles were prepared. 1.24 g of the Mn-oleate complex was dissolved in 10 g of 1-octadecene. The mixture solution was degassed at 70° C. for 1 to 2 hr under a vacuum to remove the water and oxygen. MnO nanoparticles were obtained.

A mixture of acetone and a small fraction of n-hexane were added to the solution, followed by centrifugation and washing, to yield a waxy precipitate. Thus obtained nanoparticles were re-dispersed in n-hexane, chloroform, etc. The size of the MnO nanoparticles could be controlled by varying aging time, raging from 7 nm to 35 nm (standard deviation of size variation was no more than 10%).

The colloidal stability of MnO nanoparticles with the size of 35 to nm was decreased, and precipitation by aggregation of the MnO nanoparticles sometimes occurred.

Also, the standard deviation of size variation was no more than 10%. Lastly, the MnO nanoparticles coated with typical biocompatible material, poly(ethylene glycol), were re-dispersed in water (Science, 298, p 1759, 2002) as follows: the resulting MnO nanoparticles were dispersed in chloroform (5 mg/ml) and 10 mg of 1,2-distearoyl-sn-glycero-3-phosphoethanolamine-N-[methoxy(polyethylene glycol)-2000] (mPEG-2000 PE, Avanti Polar Lipids, Inc.) was added. Chloroform was evaporated at 80° C. and then the MnO nanoparticles were re-dispersed in water.

### Example 2

### Biocompatibility and Contrast Ability of MnO Nanoparticles Coated with PEG

The sizes of nanoparticles prepared in Example 1 were very uniform and could be controllable. Also, the nanoparticles were biocompatible due to the coating with PEG, and stable over several months.

When the size of the MnO nanoparticle including a biocompatible material layer was more than 500 nm, the MnO nanoparticle coated with a biocompatible material was degraded by the immune system or in the liver, and thus residence time of the MnO nanoparticle in a living organism was decreased, resulting in decrease in MRI scanning time. Therefore, the size of the MnO nanoparticle including a biocompatible material layer should be preferably no more than 500 nm and more preferably no more than 100 nm.

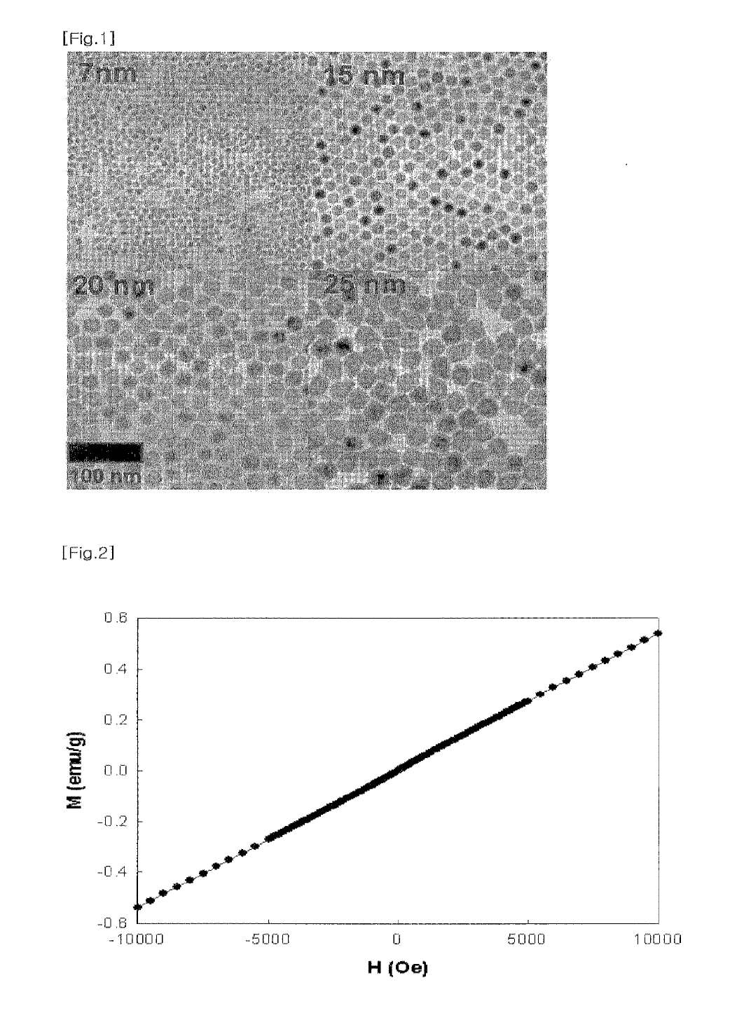

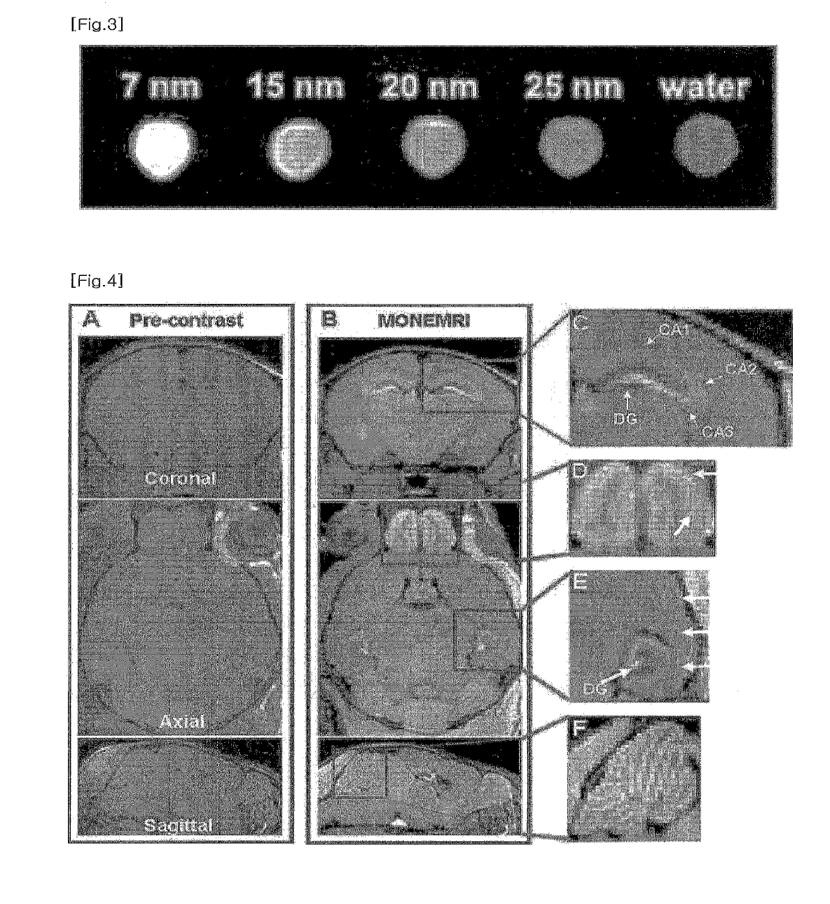

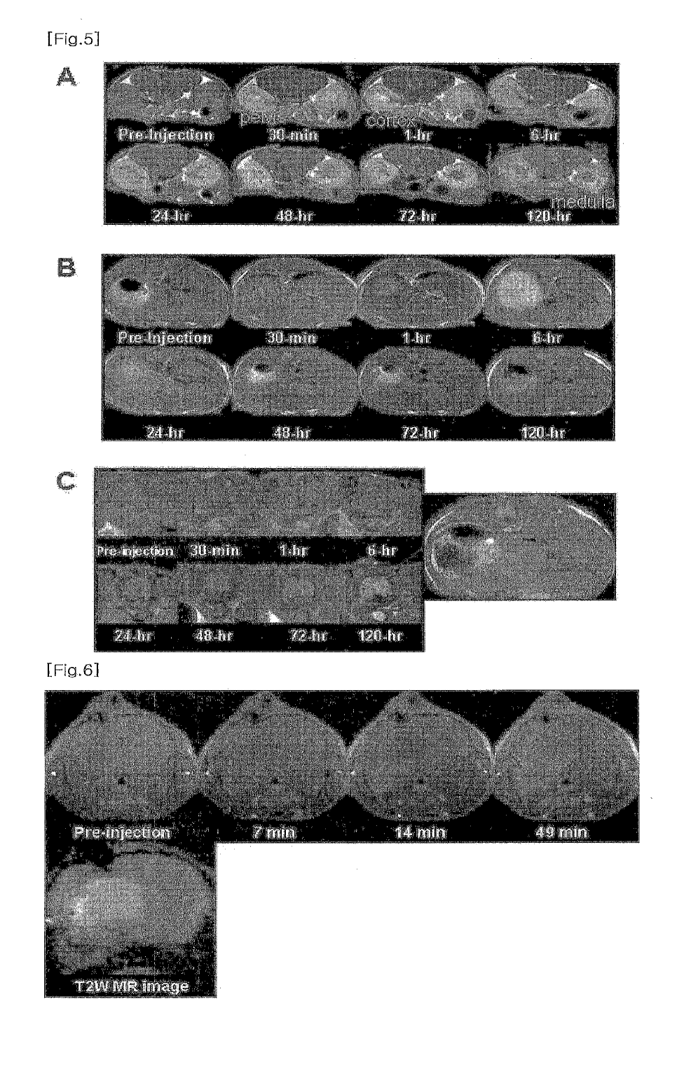

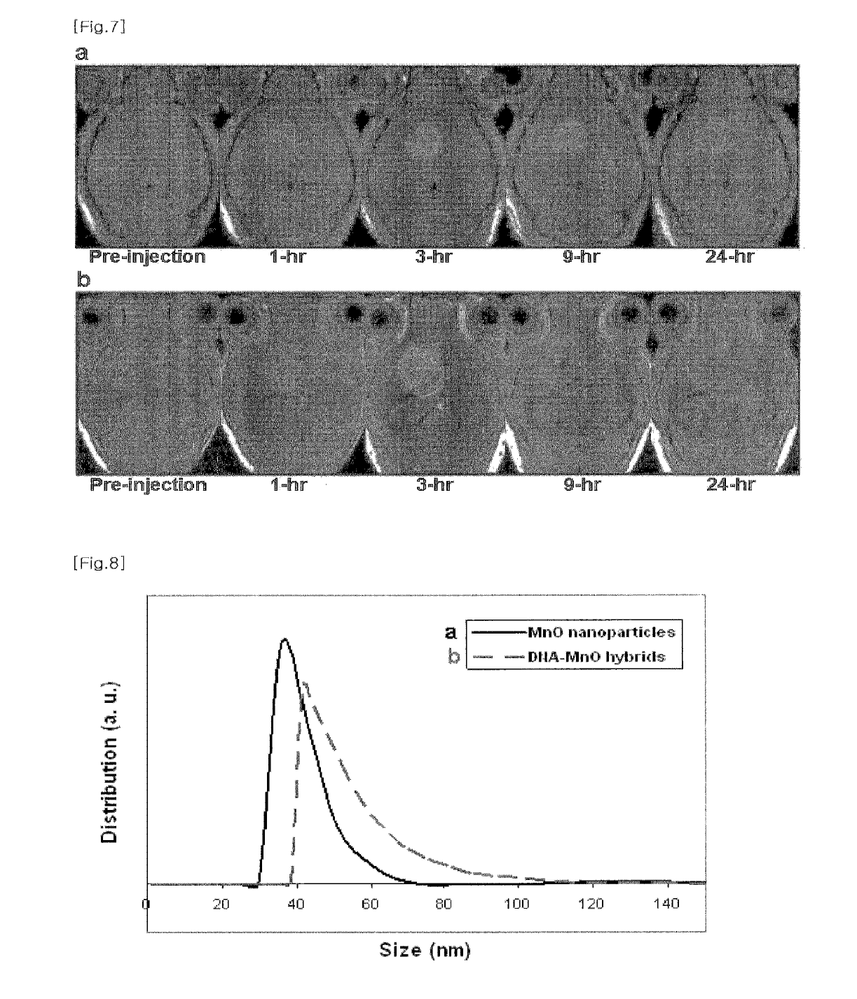

The contrast ability of MnO nanoparticles for MRI were tested with 3.0 T clinical MRI system. As shown in FIG. 2, the MnO nanoparticles at the concentration of 5 mM clearly showed bright signal enhancement in the T1 weighted MRI due to shortened T1. This manifests the contrast ability of the MnO nanoparticles as a T1 contrasting agent. Besides, T2 contrast was observed as well.

### Example 3

### Manganese Oxide Nanoparticles Enhanced MR Imaging (MONEMRI)

MONEMRI of a mouse was observed by using the MnO nanoparticles of the present invention. The MRI experiment was carried on a 4.7T/30 MRI system (Brucker-Biospin, Fallanden, Switzerland). The 25 nm sized MnO nanoparticles were bolus injected to a mouse through a tail vein, for the in vivo MRI imaging. The experimental conditions were as follows:

3-1. MRI Imaging Conditions of Brain

fast spin-echo T1-weighted MRI sequence

TR/TE=300/12.3 ms

echo train length=2

140 m 3D isotropic resolution

FOV=2.56×1.28×1.28 cm³

matrix size=256×128×128

3-2. MRI Imaging Conditions of Abdomen

fast spin-echo T1-weighted MRI sequence

TR/TE=400/12 ms

NEX=16

slice thickness=1.5 mm

FOV=2.78×168 cm²

matrix size=192×192

The resulting excellent MRI images of the mouse brain (FIG. 4) depicting fine anatomic structure were obtained, comparing with the MRI images without the contrasting agent. The excellent anatomic images of the abdomen such as kidney, liver and spinal cord were also obtained.

When the MnO nanoparticles were injected through a tail vein to a mouse bearing a gliblastoma tumor in its brain, the tumor was visualized brighter than the non-contrast enhanced images. Therefore, the cancer specific imaging was possible.

### Example 4

### Preparation of Targeting Probe Conjugated MnO Nanoparticles

Target specific probe conjugated MnO nanoparticles were prepared by the following two steps.

4.1 Synthesis of MnO Nanoparticles Having Reactive Functional Groups

At the step of coating the MnO nanoparticles dispersed in an organic solvent with biocompatible poly(ethylene glycol) in Example 1, the MnO nanoparticles were coated with phospholipids including PEG of which end was functionalized by reactive groups such as amine (—NH₂), thiol (—SH), carboxylate (—CO₂—), etc. For example, the MnO nanoparticles were coated with a mixture of 1,2-distearoyl-sn-glycero-3-phosphoethanolamine-N-[methoxy(polyethylene glycol)-2000] (mPEG-2000 PE, Avanti Polar Lipids, Inc.) and 1,2-distearoyl-sn-glycero-3-phosphoethanolamine-N-[maleimide(polyethylene glycol)-2000] (DSPE-PEG(2000) Maleimide, Avanti Polar Lipids, Inc.) in order to endow the MnO nanoparticles with maleimide. The method was similar to that of Example 1.

4.2 Preparation of the Breast Cancer Specific Antibody Conjugated MnO Nanoparticles

6 mg of Herceptin (Roche Pharma Ltd.) was dissolved in 0.5 ml of phosphate buffered saline (PBS, pH 7.2) and mixed with excess of N-succinimidyl S-acetylthioacetate (SATA). After 30 min, 0.5 M of hydroxylamine was added and the solution was incubated for 2 hr at room temperature. The resulting solution was purified with desalting column and added to 0.3 ml of maleimido-MnO (10 mg/W. It was incubated for 12 hr at 4° C. and Herceptin conjugated MnO nanoparticles were isolated through column.

### Example 5

### Cancer Specific MRI by Targeting Probe Conjugated MnO nanoparticles

The breast cancer brain metastatic tumor model was made by inoculating the MDA-MB-435 human breast cancer cells into mouse brain. The MRI examination was performed after administration of the Herceptine functionalized MnO nanoparticles. All in vivo MRI examinations were carried on a 4.7T/30 MRI system (Brucker-Biospin, Fallanden, Switzerland). The 25 nm sized water-dispersible MnO nanoparticles (35 mg of Mn measured by ICP-AES per kg of mouse body weight) were bolus (rapid single-shot) injected to a mouse through a tail vein to obtain MRIs, and the experimental conditions were similar to those of Example 3.

Thus obtained images of mouse brain are shown in FIG. 7. According to that images, Herceptin conjugated MnO nanoparticles, compared with non-functionalized MnO nanoparticles, produced more excellent cancer cell targeting MR images.

The contrasting effect was diminished after 3 hr when non-functionalized MnO nanoparticles were used. On the contrary, when Herceptin conjugated MnO nanoparticles were used, the contrasting effect was maintained even after 1 week and thus fine T1 weighted MR images were obtained. Consequently, it was easy to locate cancer cells.

### Example 6

### Oligonucleotide Conjugated MnO Nanoparticles

Amine functionalized MnO nanoparticles were prepared by the similar procedure with water dispersible MnO. To endow amine group, the mixture of 1,2-distearoyl-sn-glycero-3-phosphoethanolamine-N-[methoxy(polyethylene glycol)-2000] (mPEG-2000 PE, Avanti Polar Lipids, Inc.) and 1,2-Distearoyl-sn-Glycero-3-Phosphoethanolamine-N-[Amino(Polyethylene Glycol)2000] (DSPE-PEG(2000)Amine, Avanti Polar Lipids, Inc.) were used. MnO nanoparticles were modified by with N-succinimidyl-3-(2-pyridyldithio)-propionate (SPDP) to prepare pyridyldithiol activated MnO nanoparticles.

As a model oligonucleotide for the conjugation, the 5′ alkanethiol oligonucleotide was prepared (HS-(CH₂)₆-CGCATTCAGGAT). 0.15 nmol of pyridyldithiol activated MnO nanoparticles were mixed with 0.15 nmol 5′ alkanethiol oligonucleotide, and the solution were incubated for 12 hr at room temperature. Oligonucleotide conjugated nanoparticles were purified by centrifugal filter (MWCO: 300,000). They were characterized with dynamic light scattering and gel electroporation. Hydrodynamic diameter of resulting nanoparticles was slightly increased by the conjugation with oligonucleotides. And, due to negative charge of bound oligonucleotides, oligonucleotide conjugated MnO nanoparticles migrated faster (FIG. 9, lane 2) than the original MnO nanoparticles (FIG. 9, lane 1).

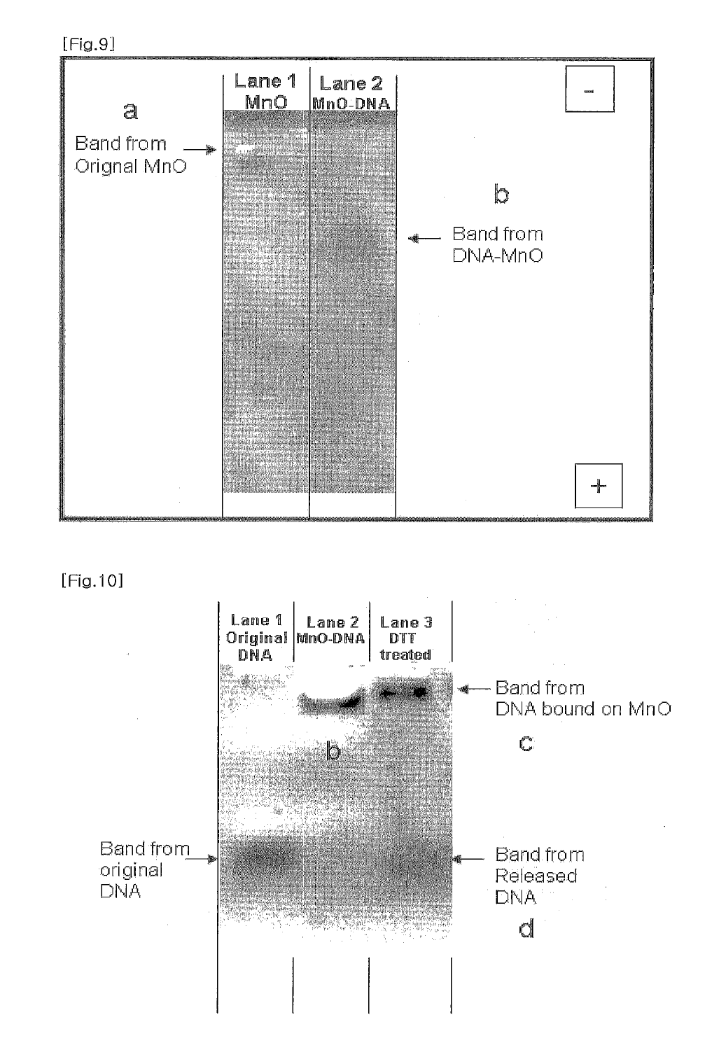

As a demonstration of the oligonucleotide delivery platform, oligonucleotides were released from these nanoparticles. 20 μl of dithiothreitol (DTT) in 10 mM PBS-EDTA buffer was mixed to 180 μl of oligonucleotide conjugated MnO nanoparticles and the solution were incubated hr at room temperature. DTT can cleave disulfide bonds and make oligonucleotides released from nanoparticles. Electrophoresis confirmed the released DNA after DTT treatment and their band (FIG. 10, lane 3) migrated as fast as the band of original oligonucleotide (FIG. 10, lane 1). On other hand, oligonucleotide conjugated MnO without DTT treatment shows much slower migration (FIG. 10, lane 2).

1: An MRI T1 contrasting agent comprising manganese oxide (MnO) nanoparticle.

2. The MRI T1 contrasting agent of claim 1, wherein said manganese nanoparticle is coated with a biocompatible material.

3. The MRI T1 contrasting agent of claim 1, wherein said biocompatible material is selected from the group consisting of polyvinyl alcohol, polylactide, polyglycolide, poly(lactide-co-glycolide), polyanhydride, polyester, polyetherester, polycaprolactone, polyesteramide, polyacrylate, polyurethane, polyvinyl fluoride, poly(vinyl imidazole), chlorosulphonate polyolefin, polyethylene oxide, poly(ethylene glycol), dextran, the mixtures thereof and the copolymers thereof.

4. The MRI T1 contrasting agent of claim 2, wherein said biocompatible material is poly(ethylene glycol).

5. The MRI T1 contrasting agent of claim 2, wherein said biocompatible material is dextran.

6. The MRI T1 contrasting agent of claim 1, wherein the diameter of said manganese oxide nanoparticle is no more than 50 nm, preferably no more than 40 nm, most preferably no more than 35 nm.

7. The MRI T1 contrasting agent of claim 1, wherein the diameter of said manganese oxide nanoparticle is no more than 30 nm.

8. The MRI T1 contrasting agent of claim 2, wherein the diameter of said MRI T1 contrasting agent comprising the biocompatible material layer is no more than 50 nm.

9. The MRI T1 contrasting agent of claim 4, wherein the thickness of said poly(ethylene glycol) layer is between 5 nm and 10 nm.

10. The MRI T1 contrasting agent of claim 6, wherein the standard deviation of diameter variation of said manganese oxide nanoparticle is no more than 10%.

11. The MRI T1 contrasting agent of claim 7, wherein the standard deviation of diameter variation of said manganese oxide nanoparticle is no more than 5%.

12. The MRI T1 contrasting agent of claim 5, wherein the diameter of said T1 contrasting agent comprising the biocompatible material layer is no more than 500 nm.

13. The MRI T1 contrasting agent of claim 1, wherein said T1 contrasting agent is a cell contrasting agent.

14. A method for preparing a MRI T1 contrasting agent, comprising:
i) thermolyzing a Mn—C₄₋₂₅ carboxylate complex to prepare a manganese nanoparticle with a diameter not exceeding 35 nm, dispersed in an organic solvent selected from the group consisting of C₆₋₂₆ aromatic hydrocarbon, C₆₋₂₆ ether, C₆₋₂₅ aliphatic hydrocarbons, C₆₋₂₆ alcohol, C₆₋₂₆ thiol, and C₆₋₂₅ amine; and
ii) coating said manganese oxide nanoparticle with a biocompatible material.

15. The method of claim 14, wherein said organic solvent of the step i) is selected from the group consisting of chloroform, 1-hexadecene and 1-octadecene.

16. The method of claim 14, wherein said biocompatible material of the step ii) is selected from the group consisting of polyvinyl alcohol, polylactide, polyglycolide, poly(lactide-co-glycolide), polyanhydride, polyester, polyetherester, polycaprolactone, polyesteramide, polyacrylate, polyurethane, polyvinyl fluoride, poly(vinyl imidazole), chlorosulphonate polyolefin, polyethylene oxide, poly(ethylene glycol), dextran, the mixtures thereof and the copolymers thereof.

17. The method of claim 14, wherein said biocompatible material is poly(ethylene glycol).

18. The method of claim 14, wherein said biocompatible material is dextran.

19. The method of claim 14, wherein the diameter of said manganese oxide nanoparticle is no more than 35 nm.

20. The method of claim 14, wherein the diameter of said manganese oxide nanoparticle is no more than 30 nm.

21. The method of claim 14, wherein the diameter of said T1 contrasting agent comprising the biocompatible material layer is no more than 500 nm.

22. The method of claim 17, wherein the thickness of said poly(ethylene glycol) layer is between 5 nm and 10 rim.

23. The method of claim 19, wherein the standard deviation of diameter variation of said manganese oxide nanoparticle is no more than 10%.

24. The method claim 20, wherein the standard deviation of diameter variation of said manganese oxide nanoparticle is no more than 5%.

25. The method of claim 18, wherein the diameter of said T1 contrasting agent comprising the biocompatible material layer is no more than 500 nm.

26. The method of claim 14, wherein said T1 contrasting agent is a cell contrasting agent.

27. An MRI T1 contrasting agent comprising manganese oxide (MnO) nanoparticle, a biocompatible material and a biologically active material, said manganese oxide nanoparticle being coated with said biocompatible material conjugated with said biologically active material.

28. The MRI T1 contrasting agent of claim 27, wherein said biologically active material is selected from the group consisting of a targeting agent selected from a protein, RNA, DNA, an antibody which selectively conjugates to a target material in a living organism, an apoptosis-inducing gene or a toxic protein; fluorescent material; isotope; a material which is sensitive to light, electromagnetic wave, radiation or heat; and a medicinally active material.

29. The MRI T1 contrasting agent of claim 27, wherein the biologically active material is selected from the group consisting of Rituxan, Herceptin, Orthoclone, Reopro, Zenapax, Synagis, Rernicade, Mylotarg, Campath, Erbitux, Avastin, Zevalin, Bexxar and the mixtures thereof.

30. The MRI T1 contrasting agent of claim 27, wherein the biologically active material is selected from the group consisting of folic acid, Vascular Endothelial Growth Factor Receptor (VEGFR), Epidermal Growth Factor Receptor (EGFR), and the ligands thereof.

31. The MRI T1 contrasting agent of claim 27, wherein the biologically active material is selected from the group consisting of amyloid beta peptide (Abeta), peptide containing RGD amino acid sequence, nuclear localization signal (NLS) peptide, TAT protein and the mixtures thereof.

32. The MRI T1 contrasting agent of claim 27, wherein the biologically active material is selected from the group consisting of cisplatin, carboplatin, procarbazine, cyclophosphamide, dactinomycin, daunorubicin, doxorubicin, bleomycin, taxol, plicomycin, mitomycin, etoposide, tamoxifen, transplatinum, vinblastin, methotrexate and the mixtures thereof.

33: The MRI T1 contrasting agent of claim 27, wherein said biocompatible material is selected from the group consisting of polyvinyl alcohol, polylactide, polyglycolide, poly(lactide-co-glycolide), polyanhydride, polyester, polyetherester, polycaprolactone, polyesteramide, polyacrylate, polyurethane, polyvinyl fluoride, poly(vinyl imidazole), chlorosulphonate polyolefin, polyethylene oxide, poly(ethylene glycol), dextran, the mixtures thereof and the copolymers thereof.

34. The MRI T1 contrasting agent of claim 27, wherein said biocompatible material is poly(ethylene glycol).

35. The MRI T1 contrasting agent of claim 27, wherein said biocompatible material is dextran.

36. The MRI T1 contrasting agent of claim 27, wherein the diameter of said manganese oxide nanoparticle is no more than 35 nm.

37. The MRI T1 contrasting agent of claim 27, wherein the diameter of said manganese oxide nanoparticle is no more than 30 nm.

38. The MRI T1 contrasting agent of claim 27, wherein the diameter of said T1 contrasting agent comprising the biologically compatible material layer is no more than 500 nm.

39. The MRI T1 contrasting agent of claim 34, wherein the thickness of said poly(ethylene glycol) layer is between 5 nm and 10 nm.

40. The MRI T1 contrasting agent of claim 36, wherein the standard deviation of diameter variation of said manganese oxide nanoparticle is no more than 10%.

41. The MRI T1 contrasting agent of claim 37, wherein the standard deviation of diameter variation of said manganese oxide nanoparticle is no more than 5%.

42. The MRI T1 contrasting agent of claim 35, wherein the diameter of said T1 contrasting agent comprising the biologically compatible material layer is no more than 500 nm.

43. The MRI T1 contrasting agent of claim 27, wherein said T1 contrasting agent is a cell contrasting agent.

44. A method for MRI T1 contrasting for animal cells using a MRT T1 contrasting agent comprising Manganese Oxide (MnO) nanoparticles.

45. A method for MRI T1 contrasting for animal blood vessels using a MRI contrasting agent comprising Manganese Oxide (MnO) nanoparticles.

46. The method of claim 44, wherein said manganese oxide nanoparticle is coated with poly(ethylene glycol).

47. The method of claim 45, wherein said manganese oxide nanoparticle is coated with dextran.
